SEQUENCE LISTING (77 sequences)

SEQ ID NO: 1 - DNA, 9432 bp, homo sapiens
gaattcaagaccagcctggacaacttggaagaacccggtctctacaaaaaatacaaaattagctgggattgggtgcggtggctcatgcctataatcccagcactttgggagcctgaggtgggtggatcacctgaagtcaggagttcaagactagcctggccaacatggtgaaaccctatctctactgaaaatacaaaaagctagacgtggtggcacacacctgtaatcccagctacttaggaggctgaggcaggagaattgcttgaagcctagaggtgaaggttgtagtgagccgagattgcatcattgcacaatggaggggagccaccagcctgggcaacaagaggaaatctccgtctccaaaaaaaaaaaaaaaaaaaaaagaattaggctgggtggtgcctgtagtcccagctacttgggaggcagggggtccacttgatgtcgagactgcagtgagccatgatcctgccactgcactccggcctgggcaacagagtgagaccctgtctaaagaaaaaaaaaataaagcaacatatcctgaacaaaggatcctccataacgttcccaccagatttctaatcagaaacatggaggccagaaagcagtggaggaggacgaccctcaggcagcccgggaggatgttgtcacaggctggggcaagggccttccggctaccaactgggagctctgggaacagccctgttgcaaacaagaagccatagcccggccagagcccaggaatgtgggctgggctgggagcagcctctggacaggagtggtcccatccaggaaacctccggcatggctgggaagtggggtacttggtgccgggtctgtatgtgtgtgtgactggtgtgtgtgagagagaatgtgtgccctaagtgtcagtgtgagtctgtgtatgtgtgaatattgtctttgtgtgggtgattttctgcgtgtgtaatcgtgtccctgcaagtgtgaacaagtggacaagtgtctgggagtggacaagagatctgtgcaccatcaggtgtgtgcatagcgtctgtgcatgtcaagagtgcaaggtgaagtgaagggaccaggcccatgatgccactcatcatcaggagctctaaggccccaggtaagtgccagtgacagataagggtgctgaaggtcactctggagtgggcaggtgggggtagggaaagggcaaggccatgttctggaggaggggttgtgactacattagggtgtatgagcctagctgggaggtggatggccgggtccactgaaaccctggttatcccagaaggctttgcaggcttcaggagcttggagtggggagagggggtgacttctccgaccaggcccctccaccggcctaccctgggtaagggcctggagcaggaagcaggggcaagaacctctggagcagcccatacccgccctggcctgactctgccactggcagcacagtcaacacagcaggttcactcacagcagagggcaaaggccatcatcagctccctttataagggaagggtcacgcgctcggtgtgctgagagtgtcctgcctggtcctctgtgcctggtggggtgggggtgccaggtgtgtccagaggagcccatttggtagtgaggcaggtatggggctagaagcactggtgcccctggccgtgatagtggccatcttcctgctcctggtggacctgatgcaccggcgccaacgctgggctgcacgctacccaccaggccccctgccactgcccgggctgggcaacctgctgcatgtggacttccagaacacaccatactgcttcgaccaggtgagggaggaggtcctggagggcggcagaggtgctgaggctcccctaccagaagcaaacatggatggtgggtgaaaccacaggctggaccagaagccaggctgagaaggggaagcaggtttgggggacgtcctggagaagggcatttatacatggcatgaaggactggattttccaaaggccaaggaagagtagggcaagggcctggaggtggagctggacttggcagtgggcatgcaagcccattgggcaacatatgttatggagtacaaagtcccttctgctgacaccagaaggaaaggccttgggaatggaagatgagttagtcctgagtgccgtttaaatcacgaaatcgaggatgaagggggtgcagtgacccggttcaaaccttttgcactgtgggtcctcgggcctcactgcctcaccggcatggaccatcatctgggaatgggatgctaactggggcctctcggcaattttggtgactcttgcaaggtcatacctgggtgacgcatccaaactgagttcctccatcacagaaggtgtgacccccacccccgccccacgatcaggaggctgggtctcctccttccacctgctcactcctggtagccccgggggtcgtccaaggttcaaataggactaggacctgtagtctggggtgatcctggcttgacaagaggccctgaccctccctctgcagttgcggcgccgcttcggggacgtgttcagcctgcagctggcctggacgccggtggtcgtgctcaatgggctggcggccgtgcgcgaggcgctggtgacccacggcgaggacaccgccgaccgcccgcctgtgcccatcacccagatcctgggtttcgggccgcgttcccaaggcaagcagcggtggggacagagacagatttccgtgggacccgggtgggtgatgaccgtagtccgagctgggcagagagggcgcggggtcgtggacatgaaacaggccagcgagtggggacagcgggccaagaaaccacctgcactagggaggtgtgagcatggggacgagggcggggcttgtgacgagtgggcggggccactgccgagacctggcaggagcccaatgggtgagcgtggcgcatttcccagctggaatccggtgtcgaagtgggggcggggaccgcacctgtgctgtaagctcagtgtgggtggcgcggggcccgcggggtcttccctgagtgcaaaggcggtcagggtgggcagagacgaggtggggcaaagcctgccccagccaagggagcaaggtggatgcacaaagagtgggccctgtgaccagctggacagagccagggactgcgggagaccagggggagcatagggttggagtgggtggtggatggtggggctaatgccttcatggccacgcgcacgtgcccgtcccacccccaggggtgttcctggcgcgctatgggcccgcgtggcgcgagcagaggcgcttctccgtgtccaccttgcgcaacttgggcctgggcaagaagtcgctggagcagtgggtgaccgaggaggccgcctgcctttgtgccgccttcgccaaccactccggtgggtgatgggcagaagggcacaaagcgggaactgggaaggcgggggacggggaaggcgaccccttacccgcatctcccacccccaggacgcccctttcgccccaacggtctcttggacaaagccgtgagcaacgtgatcgcctccctcacctgcgggcgccgcttcgagtacgacgaccctcgcttcctcaggctgctggacctagctcaggagggactgaaggaggagtcgggctttctgcgcgaggtgcggagcgagagaccgaggagtctctgcagggcgagctcccgagaggtgccggggctggactggggcctcggaagagcaggatttgcatagatgggtttgggaaaggacattccaggagaccccactgtaagaagggcctggaggaggaggggacatctcagacatggtcgtgggagaggtgtgcccgggtcagggggcaccaggagaggccaaggactctgtacctcctatccacgtcagagatttcgattttaggtttctcctctgggcaaggagagagggtggaggctggcacttggggagggacttggtgaggtcagtggtaaggacaggcaggccctgggtctacctggagatggctggggcctgagacttgtccaggtgaacgcagagcacaggagggattgagaccccgttctgtctggtgtaggtgctgaatgctgtccccgtcctcctgcatatcccagcgctggctggcaaggtcctacgcttccaaaaggctttcctgacccagctggatgagctgctaactgagcacaggatgacctgggacccagcccagcccccccgagacctgactgaggccttcctggcagagatggagaaggtgagagtggctgccacggtggggggcaagggtggtgggttgagcgtcccaggaggaatgaggggaggctgggcaaaaggttggaccagtgcatcacccggcgagccgcatctgggctgacaggtgcagaattggaggtcatttgggggctaccccgttctgtcccgagtatgctctcggccctgctcaggccaaggggaaccctgagagcagcttcaatgatgagaacctgcgcatagtggtggctgacctgttctctgccgggatggtgaccacctcgaccacgctggcctggggcctcctgctcatgatcctacatccggatgtgcagcgtgagcccatctgggaaacagtgcaggggccgagggaggaagggtacaggcgggggcccatgaactttgctgggacacccggggctccaagcacaggcttgaccaggatcctgtaagcctgacctcctccaacataggaggcaagaaggagtgtcagggccggaccccctgggtgctgacccattgtggggacgcatgtctgtccaggccgtgtccaacaggagatcgacgacgtgatagggcaggtgcggcgaccagagatgggtgaccaggctcacatgccctacaccactgccgtgattcatgaggtgcagcgctttggggacatcgtccccctgggtgtgacccatatgacatcccgtgacatcgaagtacagggcttccgcatccctaaggtaggcctggcgccctcctcaccccagctcagcaccagcacctggtgatagccccagcatggctactgccaggtgggcccactctaggaaccctggccacctagtcctcaatgccaccacactgactgtccccacttgggtggggggtccagagtataggcagggctggcctgtccatccagagcccccgtctagtggggagacaaaccaggacctgccagaatgttggaggacccaacgcctgcagggagagggggcagtgtgggtgcctctgagaggtgtgactgcgccctgctgtggggtcggagagggtactgtggagcttctcgggcgcaggactagttgacagagtccagctgtgtgccaggcagtgtgtgtcccccgtgtgtttggtggcaggggtcccagcatcctagagtccagtccccactctcaccctgcatctcctgcccagggaacgacactcatcaccaacctgtcatcggtgctgaaggatgaggccgtctgggagaagcccttccgcttccaccccgaacacttcctggatgcccagggccactttgtgaagccggaggccttcctgcctttctcagcaggtgcctgtggggagcccggctccctgtccccttccgtggagtcttgcaggggtatcacccaggagccaggctcactgacgcccctcccctccccacaggccgccgtgcatgcctcggggagcccctggcccgcatggagctcttcctcttcttcacctccctgctgcagcacttcagcttctcggtgcccactggacagccccggcccagccaccatggtgtctttgctttcctggtgagcccatccccctatgagctttgtgctgtgccccgctagaatggggtacctagtccccagcctgctccctagccagaggctctaatgtacaataaagcaatgtggtagttccaactcgggtcccctgctcacgccctcgttgggatcatcctcctcagggcaaccccacccctgcctcattcctgcttaccccaccgcctggccgcatttgagacaggggtacgttgaggctgagcagatgtcagttacccttgcccataatcccatgtcccccactgacccaactctgactgcccagattggtgacaaggactacattgtcctggcatgtggggaaggggccagaatgggctgactagaggtgtcagtcagccctggatgtggtggagagggcaggactcagcctggaggcccatatttcaggcctaactcagcccaccccacatcagggacagcagtcctgccagcaccatcacaacagtcacctcccttcatatatgacaccccaaaacggaagacaaatcatggcgtcagggagctatatgccagggctacctacctcccagggctcagtcggcaggtgccagaacgttccctgggaaggccccatggaagcccaggactgagccaccaccctcagcctcgtcacctcaccacaggactggctacctctctgggccctcagggatgctgctgtacagacccctgaccagtgacgagttcgcactcagggccaggctggcgctggaggaggacacttgtttggctccaaccctaggtaccatcctcccagtagggatcaggcagggcccacaggcctgccctagggacaggagtcaaccttggacccataaggcactggggcgggcagagaaggaggaggtggcatgggcagctgagagccagagaccctgaccctagtccttgctctgccattaccccgtgtgaccccgggcccacccttccccacccttccccaccccgggcttctgtttccttctgccaacgagaaggctgcttcacctgccccgagtcctgtcttcctgctctgccttctggggctgtggcccttgctggcctggagccccaaccaagggcagggactgctgtcctccacgtctgtcctcaccgacataatgggctgggctgggcacacaggcagtgcccaagagtttctaatgagcatatgattacctgagtcctgggcagaccttcttagggaacagcctgggacagagaaccacagacactctgaggagccaccctgaggcctcttttgccagaggaccctacagcctccctggcagcagttccgccagcatttctgtaaatgccctcatgccagggtgcggcccggctgtcagcacgagagggacgttggtctgtcccctggcaccgagtcagtcagaagggtggccagggcccccttgggcccctccagagacaatccactgtggtcacacggctcggtggcaggaagtgctgttcctgcagctgtggggacagggagtgtggatgaagccaggctgggtttgtctgaagacggaggccccgaaaggtggcagcctggcctatagcagcagcaactcttggatttattggaaagattttcttcacggttctgagtcttgggggtgttagaggctcagaaccagtccagccagagctctgtcatgggcacgtagacccggtcccagggcctttgctctttgctgtcctcagaggcctctgcaaagtagaaacaggcagccttgtgagtcccctcctgggagcaaccaaccctccctctgagatgccccggggccaggtcagctgtggtgaaaggtagggatgcagccagctcagggagtggcccagagttcctgcccacccaaggaggctcccaggaaggtcaaggcacctgactcctgggctgcttccctcccctcccctccccaggtcaggaaggtgggaaagggctggggtgtctgtgaccctggcagtcactgagaagcagggtggaagcagccccctgcagcacgctgggtcagtggtcttaccagatggatacgcagcaacttccttttgaacctttttattttcctggcaggaagaagagggatccagcagtgagatcaggcaggttctgtgttgcacagacagggaaacaggctctgtccacacaaagtcggtggggccaggatgaggcccagtctgttcacacatggctgctgcctctcagctctgcacagacgtcctcgctcccctgggatggcagcttggcctgctggtcttggggttgagccagcctccagcactgcctccctgccctgctgcctcccactctgcagtgctccatggctgctcagttggacccacgctggagacgttcagtcgaagccccgggctgtccttacctcccagtctggggtacctgccacctcctgctcagcaggaatggggctaggtgcttcctcccctggggacttcacctgctctccctcctgggataagacggcagcctcctccttgggggcagcagcattcagtcctccaggtctcctgggggtcgtgacctgcaggaggaataagagggcagactgggcagaaaggccttcagagcacctcatcctcctgttctcacactggggtgtcacagtcctgggaagttcttccttttcagttgagctgtggtaaccttgtgagtttcctggagggggcctgccactacccttgggactccctgccgtgtgtctgggtctaactgagctctgaaaggagagagccccagccctgggccttccaggggaagccttacctcagaggttggcttcttcctactcttgactttgcgtctctgcagagggaggtgggaggggtgacacaaccctgacacccacactatgagtgatgagtagtcctgccccgactggcccatcctttccaggtgcagtcccccttactgtgtctgccaagggtgccagcacagccgccccactccaggggaagaggagtgccagcccttaccacctgagtgggcacagtgtagcatttattcattagcccccacactggcctgaccatctcccctgtgggctgcatgacaaggagagagaacaggctgaggtgagagctactgtcaacacctaaacctaaaaaatctataattgggctgggcagggtggctcacgcctgtaatcccagcactttgggaggccgagatgggtggatcacctgaggtcagatgttcgagaccagcctggccaacatggtgaaaccccgtctctactaaaaatacaaaaaattagctgggcgtggtggtgggtgcctgtaatcccagctactcaggaggctgaggcaggagaattgcttgaacctgggaggcagaggctgcagtgagccgagatcgcatcattgcactccagcctggtcaacaagagtgaaactgtcttaaaaaaaaaatctataattgatatctttagaaagataaaactttgcattcatgaaataagaataggagggtctaaaataaaaatgttcaaacacccaccaccactaattcttgacaaaaatatagtctgggtgccttagctcatgcctgtaatcccagcattttgggaggctaaggcaggaggattgtttgagcctaggaattc

SEQ ID NO: 2 - DNA, 1680 bp, homo sapiens
gaattcaagaccagcctggacaacttggaagaaccsggtctctacaaaaaatacaaaattagctgggattgggtgcggtggctcatgcctataatcccagcactttgggagcctgaggtgggtggatcacctgaagtcaggagttcaagactagcctggccaacatggtgaaaccctatctctactgaaaatayaaaaagctagacgtggtggcacacacctgtaatcccagctacttaggaggctgaggcaggagaattgcttgaagcctagaggtgaaggttgtagtgagccgagattgcatcattgcacaatggaggggagccaccagcctgggcaacaagaggaaatctccgtctccaaaaaaaaaaaaaaaaaaaaaagrattaggctgggtggtgcctgtagtcccagctacttgggaggcagggggtccacttgatgtcgagactgcagtgagccatgatcctgccactgcactccggcctgggcaacagagtgagaccctgtctaaagaaaaaaaaaataaagcaacatatcctgaacaaaggatcctccataacgttcccaccagatttctaatcagaaacatggaggccagaaagcagtggaggaggacraccctcaggcagcccgggaggatgttgtcacaggctggggcaagggccttccggctaccaactgggagctctgggaacagccctgttgcaaacaagaagccatagcccggccagagcccaggaatgtgggctgggctgggagcagcctctggacaggagtggtcccatccaggaaacctccggcatggctgggaagtggggtacttggtgccgggtctgtatgtgtgtgtgactggtgtgtgtgagagagaatgtgtgcyctaagtgtcagtgtgagtctgtgtatgtgtgaatattgtctttgtgtgggtgattttctgcrtgtgtaatcgtgtccctgcaagtgtgaacaagtggacaagtgtctgggagtggacaagagatctgtgcaccatcaggtgtgtgcatagcgtctgtgcatgtcaagagtgcaaggtgaagtgaagggaccaggcccatgatgccactcatcatcaggagctctaaggccccaggtaagtgccagtgacagataagggtgctgaaggtcactctggagtgggcaggtgggggtagggaaagggcaaggccatgttctggaggaggggttgtgactacattagggtgtatgagcctagctgggaggtggatggccrggtccactgaaaccctggttatcccagaaggctttgcaggcttcaggagcttggagtggggagagggggtgacttctccgaccaggcccctccaccggcctaccctgggtaagggcctggagcaggaagcaggggcaagaacctctggagcagcccatacccgccctggcctgactctgccactggcagcacagtcaacacagcaggttcactcacagcagagggcaaaggccatcatcagctccctttataagggaagggtcacgcgctcggtgtgctgagagtgtcctgcctggtcctctgtgcctggtggggtgggggtgccaggtgtgtccagaggagcccatttggtagtgaggcaggtatggggctagaagcactggtgcccctggccgtgatagtggccatcttcctgctcctggtgg

SEQ ID NO: 3 - DNA, 11 bp, Artificial sequence
  synthetic oligonucleotide
gaacccggtct

SEQ ID NO: 4 - DNA, 13 bp, artificial sequence
  synthetic oligonucleotide
aaaatacaaaaag

SEQ ID NO: 5 - DNA, 13 bp, artificial sequence
  synthetic oligonucleotide
aaaaagaattagg

SEQ ID NO: 6 - DNA, 11 bp, artificial sequence
  synthetic oligonucleotide
aggacgaccct

SEQ ID NO: 7 - DNA, 11 bp, artificial sequence
  synthetic oligonucleotide
tgtgccctaag

SEQ ID NO: 8 - DNA, 11 bp, artificial sequence
  synthetic oligonucleotide
tctgcgtgtgt

SEQ ID NO: 9 - DNA, 11 bp, artificial sequence
  synthetic oligonucleotide
tggccgggtcc

SEQ ID NO: 10 - DNA, 11 bp, artificial sequence
  synthetic oligonucleotide
gaaccgggtct

SEQ ID NO: 11 - DNA, 13 bp, artificial sequence
  synthetic oligonucleotide
aaaatataaaaag

SEQ ID NO: 12 - DNA, 13 bp, artificial sequence
  synthetic oligonucleotide
aaaaaggattagg

SEQ ID NO: 13 - DNA, 11 bp, artificial sequence
  synthetic oligonucleotide
aggacaaccct

SEQ ID NO: 14 - DNA, 11 bp, artificial sequence
  synthetic oligonucleotide
tgtgctctaag

SEQ ID NO: 15 - DNA, 11 bp, artificial sequence
  synthetic oligonucleotide
tctgcatgtgt

SEQ ID NO: 16 - DNA, 11 bp, artificial sequence
  synthetic oligonucleotide
tggccaggtcc

SEQ ID NO: 17 - DNA, 23 bp, artificial sequence
  synthetic oligonucleotide
aaatacaaaattagctgggattg

SEQ ID NO: 18 - DNA, 21 bp, artificial sequence
  synthetic oligonucleotide
gagacggagatttcctcttgt

SEQ ID NO: 19 - DNA, 18 bp, artificial sequence
  synthetic oligonucleotide
ccttccggctaccaactg

SEQ ID NO: 20 - DNA, 20 bp, artificial sequence
  synthetic oligonucleotide
ttgcagggacacgattacac

SEQ ID NO: 21 - DNA, 21 bp, artificial sequence
  synthetic oligonucleotide
taagggtgctgaaggtcactc

SEQ ID NO: 22 - DNA, 18 bp, artificial sequence
  synthetic oligonucleotide
gggctgctccagaggttc

SEQ ID NO: 23 - DNA, 20 bp, artificial sequence
  synthetic oligonucleotide
ccaggtaagtgccagtgaca

SEQ ID NO: 24 - DNA, 20 bp, artificial sequence
  synthetic oligonucleotide
agctcctgaagcctgcaaag

SEQ ID NO: 25 - DNA, 18 bp, artificial sequence
  synthetic oligonucleotide
gccagagcccaggaatgt

SEQ ID NO: 26 - DNA, 18 bp, artificial sequence
  synthetic oligonucleotide
gccttgccctttccctac

SEQ ID NO: 27 - DNA, 19 bp, artificial sequence
  synthetic oligonucleotide
agaaacatggaggccagaa

SEQ ID NO: 28 - DNA, 19 bp, artificial sequence
  synthetic oligonucleotide
gtttcctggatgggaccac

SEQ ID NO: 29 - DNA, 22 bp, artificial sequence
  synthetic oligonucleotide
agcctagaggtgaaggttgtag

SEQ ID NO: 30 - DNA, 17 bp, artificial sequence
  synthetic oligonucleotide
cttgccccagcctgtga

SEQ ID NO: 31 - DNA, 25 bp, artificial sequence
  synthetic oligonucleotide
aaaaaatacaaaattagctgggatt

SEQ ID NO: 32 - DNA, 21 bp, artificial sequence
  synthetic oligonucleotide
tttttttttggagacggagat

SEQ ID NO: 33 - DNA, 23 bp, artificial sequence
  synthetic oligonucleotide
ttctttagacagggtctcactct

SEQ ID NO: 34 - DNA, 19 bp, artificial sequence
  synthetic oligonucleotide
gggcaacaagaggaaatct

SEQ ID NO: 35 - DNA, 19 bp, artificial sequence
  synthetic oligonucleotide
gcctggacaacttggaaga

SEQ ID NO: 36 - DNA, 11 bp, artificial sequence
  synthetic oligonucleotide
agaccgggttc

SEQ ID NO: 37 - DNA, 11 bp, artificial sequence
  synthetic oligonucleotide
agacccggttc

SEQ ID NO: 38 - DNA, 13 bp, artificial sequence
  synthetic oligonucleotide
ctttttgtatttt

SEQ ID NO: 39 - DNA, 13 bp, artificial sequence
  synthetic oligonucleotide
ctttttatatttt

SEQ ID NO: 40 - DNA, 13 bp, artificial sequence
  synthetic oligonucleotide
cctaattcttttt

SEQ ID NO: 41 - DNA, 13 bp, artificial sequence
  synthetic oligonucleotide
cctaatccttttt

SEQ ID NO: 42 - DNA, 11 bp, artificial sequence
  synthetic oligonucleotide
agggtcgtcct

SEQ ID NO: 43 - DNA, 11 bp, artificial sequence
  synthetic oligonucleotide
agggttgtcct

SEQ ID NO: 44 - DNA, 11 bp, artificial sequence
  synthetic oligonucleotide
cttagggcaca

SEQ ID NO: 45 - DNA, 11 bp, artificial sequence
  synthetic oligonucleotide
cttagagcaca

SEQ ID NO: 46 - DNA, 11 bp, artificial sequence
  synthetic oligonucleotide
acacacgcaga

SEQ ID NO: 47 - DNA, 11 bp, artificial sequence
  synthetic oligonucleotide
acacatgcaga

SEQ ID NO: 48 - DNA, 11 bp, artificial sequence
  synthetic oligonucleotide
ggacccggcca

SEQ ID NO: 49 - DNA, 11 bp, artificial sequence
  synthetic oligonucleotide
ggacctggcca

SEQ ID NO: 50 - DNA, 11 bp, artificial sequence
  synthetic oligonucleotide
acttggaagaa

SEQ ID NO: 51 - DNA, 11 bp, artificial sequence
  synthetic oligonucleotide
tctactgaaaa

SEQ ID NO: 52 - DNA, 25 bp, artificial sequence
  synthetic oligonucleotide
ccaaaaaaaaaaaaaaaaaaaaaag

SEQ ID NO: 53 - DNA, 11 bp, artificial sequence
  synthetic oligonucleotide
agtggaggagg

SEQ ID NO: 54 - DNA, 11 bp, artificial sequence
  synthetic oligonucleotide
agagaatgtgt

SEQ ID NO: 55 - DNA, 11 bp, artificial sequence
  synthetic oligonucleotide
ggtgattttct

SEQ ID NO: 56 - DNA, 11 bp, artificial sequence
  synthetic oligonucleotide
gaggtggatgg

SEQ ID NO: 57 - DNA, 11 bp, artificial sequence
  synthetic oligonucleotide
ttcttccaagt

SEQ ID NO: 58 - DNA, 11 bp, artificial sequence
  synthetic oligonucleotide
ttttcagtaga

SEQ ID NO: 59 - DNA, 25 bp, artificial sequence
  synthetic oligonucleotide
cttttttttttttttttttttttgg

SEQ ID NO: 60 - DNA, 11 bp, artificial sequence
  synthetic oligonucleotide
cctcctccact

SEQ ID NO: 61 - DNA, 11 bp, artificial sequence
  synthetic oligonucleotide
acacattctct

SEQ ID NO: 62 - DNA, 11 bp, artificial sequence
  synthetic oligonucleotide
agaaaatcacc

SEQ ID NO: 63 - DNA, 11 bp, artificial sequence
  synthetic oligonucleotide
ccatccacctc

SEQ ID NO: 64 - DNA, 11 bp, artificial sequence
  synthetic oligonucleotide
ttttgtagaga

SEQ ID NO: 65 - DNA, 11 bp, artificial sequence
  synthetic oligonucleotide
cgtctagcttt

SEQ ID NO: 66 - DNA, 11 bp, artificial sequence
  synthetic oligonucleotide
cacccagccta

SEQ ID NO: 67 - DNA, 11 bp, artificial sequence
  synthetic oligonucleotide
gctgcctgagg

SEQ ID NO: 68 - DNA, 11 bp, artificial sequence
  synthetic oligonucleotide
cactgacactt

SEQ ID NO: 69 - DNA, 11 bp, artificial sequence
  synthetic oligonucleotide
acacgattaca

SEQ ID NO: 70 - DNA, 11 bp, artificial sequence
  synthetic oligonucleotide
gtttcagtgga

SEQ ID NO: 71 - DNA, 11 bp, artificial sequence
  synthetic oligonucleotide
tctctacaaaa

SEQ ID NO: 72 - DNA, 11 bp, artificial sequence
  synthetic oligonucleotide
aaagctagacg

SEQ ID NO: 73 - DNA, 11 bp, artificial sequence
  synthetic oligonucleotide
taggctgggtg

SEQ ID NO: 74 - DNA, 11 bp, artificial sequence
  synthetic oligonucleotide
cctcaggcagc

SEQ ID NO: 75 - DNA, 11 bp, artificial sequence
  synthetic oligonucleotide
aagtgtcagtg

SEQ ID NO: 76 - DNA, 11 bp, artificial sequence
  synthetic oligonucleotide
tgtaatcgtgt

SEQ ID NO: 77 - DNA, 11 bp, artificial sequence
  synthetic oligonucleotide
tccactgaaac

(19) United States
(12) Patent Application Publication
(10) Pub. No.: US 2003/0044797 A1
(43) Pub. Date: Mar. 6, 2003

(54) Detection of CYP2D6 polymorphisms

(76) Inventors: Carl Risinger, Uppsala (SE); Maria Kristina Andersson, Uppsala (SE); Tommy Lewander, Uppsala (SE); Erik Olaisson, Bonadsvagen (SE)

Correspondence Address: MORRISON & FOERSTER LLP, 3811 VALLEY CENTRE DRIVE, SUITE 500, SAN DIEGO, CA, 92130-2332, US

(21) Appl. No.: 09/942,310

(22) Filed: Aug. 29, 2001

(30) Foreign Application Priority Data
Aug. 30, 2000 (GB) 0021286.0

(51) Int. Cl. C12Q 1/68; C07H 21/04

(52) U.S. Cl. 435/6; 536/23.2

(57) ABSTRACT

The invention provides methods, PCR primers, sequence determination oligonucleotides, and kits for predicting a human's capacity to metabolize a substrate of the CYP2D6 enzyme using genetic analysis.

The present invention is directed to detection of certain polymorphisms in the 5′ regulatory region of the gene encoding cytochrome P450 2D6, also known as CYP2D6, bufuralol-1′-hydroxylase, or debrisoquine/sparteine hydroxylase, to predict variations in an individual's ability to metabolize certain drugs.

### BACKGROUND OF THE INVENTION

Xenobiotics are pharmacologically, endocrinologically, or toxicologically active substances foreign to a biological system. Most xenobiotics, including pharmaceutical agents, are metabolized through two successive reactions. Phase I reactions (functionalization reactions), include oxidation, reduction, and hydrolysis, in which a derivatizable group is added to the original molecule. Functionalization prepares the drug for further metabolism in phase II reactions. During phase II reactions (conjugative reactions, which include glucoronidation, sulfation, methylation and acetylation), the functionalized drug is conjugated with a hydrophilic group. The resulting hydrophilic compounds are inactive and excreted in bile or urine. Thus, metabolism can result in detoxification and excretion of the active substance. Alternatively, an inert xenobiotic may be metabolized to an active compound. For example, a pro-drug may be converted to a biologically active therapeutic or toxin.

The cytochrome P450 (CYP) enzymes are involved in the metabolism of many different xenobiotics. CYPs are a superfamily of heme-containing enzymes, found in eukaryotes (both plants and animals) and prokaryotes, and are responsible for Phase I reactions in the metabolic process. In total, over 500 genes belonging to the CYP superfamily have been described and divided into subfamilies, CYP1-CYP27. In humans, more than 35 genes and 7 pseudogenes have been identified. Members of three CYP gene families, CYP1, CYP2, and CYP3, are responsible for the majority of drug metabolism. The human CYPs which are of greatest clinical relevance for the metabolism of drugs and other xenobiotics are CYP1A2, CYP2A6, CYP2C9, CYP2C19, CYP2D6, CYP2E1 and CYP3A4. The liver is the major site of activity of these enzymes, however CYPs are also expressed in other tissues.

Approximately 20% of known drugs are substrates for CYP2D6, and thus the metabolism of these drugs is wholly or partially mediated by this enzyme. For example, a variety of antidepressants including the tricyclic antidpressants and the SSRIs (serotonin reuptake inhibitors) are substrates of CYP2D6. Antipsychotics such as haloperidol, perphenazine, thioridazine, and zuclopenthixol are also substrates of CYP2D6. In addition, CYP2D6 hydroxylates β-adrenoreceptor blocking agents such as propranolol, metoprolol, and timolol and anti-arrhythmic drugs such as sparteine, diprafenone, and propafenone. Codeine is hydroxylated to morphine by CYP2D6.

CYP2D6 is a polymorphic enzyme, that is, more than one form of the enzyme is present within the human population. The different forms of the CYP2D6 enzyme have differing abilities to hydroxylate substrates, which impacts on the rate at which the substrates are removed from the body. The form of CYP2D6 that an individual inherits will determine how quickly a substrate is removed from the individual's body. Because CYP2D6 is polymorphic, individuals differ in their ability to metabolize the drugs that are substrates of CYP2D6, and consequently, wide variations in responses to such drugs, including susceptibility to side effects, have been observed.

On the basis of ability of metabolize a marker drug such as debrisoquine or sparteine, individuals may be characterized as poor metabolizers (PM), intermediate metabolizers (IM), extensive metabolizers (EM) or ultra extensive metabolizers (UEM or UM) for CYP2D6 substrates. Poor metabolizers retain the CYP2D6 substrate in their bodies for a relatively long period of time, and are susceptible to toxicity and side effects at “normal” dosages. Ultraextensive metabolizers clear the CYP2D6 substrate from their bodies quickly, and require higher than “normal” dosages to achieve a therapeutic effect. Intermediate and extensive metabolizers retain the CYP2D6 substrate in their bodies for times between those of PMs and UEMs, and are more likely to respond to “normal” dosages of the drug. However, individuals characterized as IM or EM may differ in drug clearance by as much as 80-fold, and variations in toxicity, side effects, and efficacy for a particular drug may occur among these individuals.

The existence of more than one form of the CYP2D6 enzyme is caused by polymorphisms in the gene which encodes the CYP2D6 enzyme (the gene being denoted in italics, as CYP2D6, SEQ ID NO: 1). In fact, more than 30 polymorphisms in the CYP2D6 gene have been described (see http://www.imm.ki.se/cypalleles/for listing). The frequency of a particular CYP2D6 polymorphism may differ widely among ethnic groups, with concomitant differences in CYP2D6 activity and responses to drugs which are CYP2D6 substrates. The frequencies of CYP2D6 mutations in European populations are presented in Marez, et al. (1997) Pharmacogenetics 7, 193-202 and Sachse, et al. (1997) Am. J. Hum. Genet. 60, 284-295. The most common polymorphisms are CYP2D6*1A, CYP2D6*2, CYP2D6*2B, CYP2D6*4A, and CYP2D6*5, which account for about 87% of all CYP2D6 alleles in Europeans. CYP2D6*1A encodes an active enzyme and is commonly known as the wild type gene. CYP2D6*2 and CYP2D6*2B encode a functional enzyme which has slightly decreased activity. CYP2D6*4A includes a G to A substitution at position 3465 of SEQ ID NO:1, which results in a splicing defect and a truncated, inactive protein, and CYP2D6*5 is a deletion of the entire CYP2D6 gene, resulting in no CYP2D6 enzyme activity.

Polymorphisms which result in a defective or absent CYP2D6 enzyme are generally correlated with the PM phenotype. A number of efforts have been made to detect these polymorphisms in order to predict an individual's response to CYP2D6 substrates without administering a potentially toxic drug. For example, WO 91/10745 discloses a method of identifying mutations at one or more of positions 100, 271, 281, 294, 408, 506, 775, or 1432 of CYP2D6, to distinguish PMs from EMs. The numbering of the CYP2D6 sequence employed in WO 91/10745 began at the initiation codon and thus did not include the 5′ flanking region of the gene.

U.S. Pat. No. 5,648,482 and corresponding EP 463 395 B1 disclose polymerase chain reaction (PCR) primers for specifically amplifying alleles of the CYP2D6 gene, for detection of PMs. The PCR primers of U.S. Pat. No. 5,648,482 and EP 463 395 B1 are complementary to intronic sequences unique to CYP2D6.

EP 759 476 A1 discloses PCR primers and methods for detecting a nine base pair insertion in exon 9 of CYP2D6, useful for detecting PMs.

The UEM phenotype is generally correlated with amplifications of functional CYP2D6 genes. Bertilsson et al. (1996) CNS Drugs 5, 200-223 discloses that such amplifications include duplications and triplications, though up to 13 copies of the CYP2D6 gene have been found in some families. Lundqvist, et al. (1999) Gene 226, 327-338, discloses that 7% of Spaniards, 29% of Ethiopians, and 20% of Saudi Arabians have duplicated or multiduplicated CYP2D6 genes. Bertilsson et al. also discloses that a duplicated or amplified CYP2D6L2 (CYP2D6*2XN) allele is present in about 1-2% of the Swedish Caucasian population, and that this allele is present only in about 40% of individuals with a metabolic ratio of less than 0.1. Thus CYP2D6 gene amplification may not explain the genetics of all CYP2D6 UEM, and additional methods of detecting such individuals are needed.

Lundqvist, et al. discloses several mutations in the 5′ flanking region of the CYP2D6 gene, including a C to G substitution at −1496, a 5A insertion between −1149 and −1148, an A to G substitution at −1147, a C to T substitution at −653, and a G to A substitution at −591. Raimundo, et al. (1999) Eur. J. Clin. Pharmacol. 55, A5 discloses seven point mutations in the 5′ flanking region of the CYP2D6 gene in an abstract describing a study to characterize inter-individual metabolic capacity in EMs. The mutations disclosed were: −234 (C to T), −590 (A to G), −652 (T to C), −912 (A to G), −1147(G to A), −1338 (T to C), and −1496(G to C). In a later publication, Raimundo, et al. (2000) Pharmacogenetics 10, 577-581, the mutations at −1496, −652, and −590 were disclosed to be exclusively associated with the functional CYP2D6*2 allele, and the mutations at −1338 and −912 were disclosed to be associated with the nonfunctional CYP2D6*4 allele and the functional CYP2D6*10 allele. The mutation at −1147 was found in all alleles investigated. On the basis of an association between the mutation at −1496 and the EM phenotype, in particular for individuals having the CYP2D6*2/CYP2D6*0 genotype, Raimundo et al. proposed that that at least 50-60% of all IMs could be predicted.

U.S. Pat. No. 6,045,996 discloses oligonucleotide arrays including the complete coding sequence of the CYP2D6 gene, exon by exon, including probes to detect specifically known polymorphisms.

Because of the complexity of the CYP2D6 genetic locus and the impact of the CYP2D6 enzyme on drug metabolism, additional diagnostic or prognostic methods and tools are needed. Such methods and tools will be useful in predicting an individual's likely response to a drug and in selecting subjects for clinical trials.

### SUMMARY OF THE INVENTION

The present inventors have discovered that individuals who are homozygous or heterozygous for certain haplotypes consisting of polymorphic sites in the 5′ flanking region of the CYP2D6 gene exhibit characteristic metabolic ratios for debrisoquine. Using this information, the capacity of individuals to metabolize drugs which are substrates of the CYP2D6 enzyme may be predicted by genotyping those polymorphisms.

In one embodiment, the invention provides a method for determining a human's capacity to metabolize a substrate of a CYP2D6 enzyme, said method comprising the steps of: isolating single stranded nucleic acids from the human, said nucleic acids encoding 5′ flanking regions of CYP2D6 genes present on each homologous chromosome 22 of the human, wherein said region is represented by a sequence as set forth in SEQ ID NO:2; and detecting at least three polymorphisms within the region, wherein the polymorphisms are selected from the group consisting of nucleotides present at polymorphic sites represented by positions 36, 194, and 942 of SEQ ID NO:2; nucleotides at polymorphic sites represented by positions 36, 620, and 942 of SEQ ID NO:2; nucleotides at polymorphic sites represented by positions 36, 194, and 880 of SEQ ID NO:2; nucleotides at polymorphic sites represented by positions 36, 620, and 880 of SEQ ID NO:2; nucleotides at polymorphic sites represented by positions 36, 194, 620, and 880 of SEQ IDNO:2; nucleotides at polymorphic sites represented by positions 36 , 194 , 620 , and 942 of SEQ ID NO:2; nucleotides at polymorphic sites represented by positions 36, 620, 880, and 942 of SEQ ID NO:2; and nucleotides at polymorphic sites represented by positions 36, 194, 620, 880, and 942 of SEQ ID NO:2.

In another embodiment, the invention provides a sequence determination oligonucleotide suitable for detecting polymorphic sites in a 5′ flanking region of a CYP2D6 gene, said oligonucleotide comprising a sequence selected from the group consisting of a sequence complementary to the polymorphic region corresponding to position 36 of SEQ ID NO:2; a sequence complementary to the polymorphic region corresponding to position 194 of SEQ ID NO:2; a sequence complementary to the polymorphic region corresponding to position 620 of SEQ ID NO:2; a sequence complementary to the polymorphic region corresponding to position 880 of SEQ ID NO:2; and a sequence complementary to the polymorphic region corresponding to position 942 of SEQ ID NO:2.

In another embodiment, the invention provides an oligonucleotide primer pair suitable for amplifying a 5′ flanking region of a CYP2D6 gene, said primer pair comprising sequences selected from the group consisting of: SEQ ID NO:17 and SEQ ID NO:18; SEQ ID NO:19 and SEQ ID NO:20; SEQ ID NO:21 and SEQ ID NO:22; SEQ ID NO:23 and SEQ ID NO:24; SEQ ID NO:25 and SEQ ID NO:26; SEQ ID NO:27 and SEQ ID NO:28; SEQ ID NO:29 and SEQ ID NO:30; SEQ ID NO:31 and SEQ ID NO:32; SEQ ID NO:33 and SEQ ID NO:34; and SEQ ID NO:35 and SEQ ID NO:18.

In another embodiment, the invention provides a kit comprising at least three pairs of oligonucleotide primers suitable for amplifying a 5′ flanking region of a CYP2D6 gene, said primer pairs being selected from the group consisting of SEQ ID NO:17 and SEQ ID NO:18; SEQ ID NO:19 and SEQ ID NO:20; SEQ ID NO:21 and SEQ ID NO:22; SEQ ID NO:23 and SEQ ID NO:24; SEQ I D NO:25 and SEQ ID NO: 26; SEQ ID NO:27 and SEQ ID NO: 28; SEQ ID NO:29 and SEQ ID NO:30; SEQ ID NO:31 and SEQ ID NO:32; SEQ ID NO:33 and SEQ ID NO:34; and SEQ ID NO:35 and SEQ ID NO:18; and at least three sequence determination oligonucleotides, said oligonucleotides comprising sequences selected from the group consisting of: SEQ ID NO:3; SEQ ID NO:10; SEQ ID NO:36; SEQ ID NO:37; SEQ ID NO:50; SEQ ID NO:57; SEQ ID NO:64; SEQ ID NO:71 (for PCR-products amplified with SEQ ID NO:35 and SEQ ID NO:18) for polymorphic position 36; SEQ ID NO:4; SEQ ID NO: 1; SEQ ID NO:38; SEQ ID NO:39; SEQ ID NO:51; SEQ ID NO:58; SEQ ID NO:65; SEQ ID NO:72 (for PCR-products amplified with SEQ ID NO: 17 and SEQ ID NO:18, or SEQ ID NO:31 and SEQ ID NO:32) for polymorphic position 194; SEQ ID NO:5; SEQ ID NO: 12; SEQ ID NO:40; SEQ ID NO:41; SEQ ID NO:52; SEQ ID NO:59; SEQ ID NO:66; SEQ ID NO:73 (for PCR-products amplified with SEQ ID NO:29 and SEQ ID NO:30, or SEQ ID NO:33 and SEQ ID NO:34) for polymorphic position 385; SEQ ID NO:6; SEQ ID NO:13; SEQ ID NO:42; SEQ ID NO:43; SEQ ID NO:53; SEQ ID NO:60; SEQ ID NO:67; SEQ ID NO:74 (for PCR-products amplified with SEQ ID NO:27 and SEQ ID NO:28, or SEQ ID NO:29 and SEQ ID NO:30) for polymorphic position 620; SEQ ID NO:7; SEQ ID NO: 14; SEQ ID NO:44; SEQ ID NO:45; SEQ ID NO:54; SEQ ID NO:61; SEQ ID NO:68; SEQ ID NO:75 (for PCR-products amplified with SEQ ID NO:19 and SEQ ID NO:20, or SEQ ID NO:25 and SEQ ID NO:26) for polymorphic position 880; SEQ ID NO:8; SEQ ID NO:15; SEQ ID NO:46; SEQ ID NO:47; SEQ ID NO:55; SEQ ID NO:62; SEQ ID NO:69; SEQ ID NO:76 (for PCR-products amplifled with SEQ ID NO:19 and SEQ ID NO:20, or SEQ ID NO:25 and SEQ ID NO:26) for polymorphic position 942; and SEQ ID NO:9; SEQ ID NO:16; SEQ ID NO:48; SEQ ID NO:49; SEQ ID NO:56; SEQ ID NO:63; SEQ ID NO:70; SEQ ID NO:77 (for PCR-products amplified with SEQ ID NO:21 and SEQ ID NO:22, or SEQ ID NO:23 and SEQ ID NO:24) for polymorphic position 1255.

### BRIEF DESCRIPTION OF THE DRAWINGS

FIG. 1 shows the sequence of the CYP2D6 gene as set forth in SEQ ID NO:1.

FIG. 2 shows the 5′ flanking region of the CYP2D6 gene as set forth in SEQ ID NO:2 with polymorphic sites highlighted in bold.

FIG. 3 outlines the One Base Sequencing (OBS) method of SNP detection.

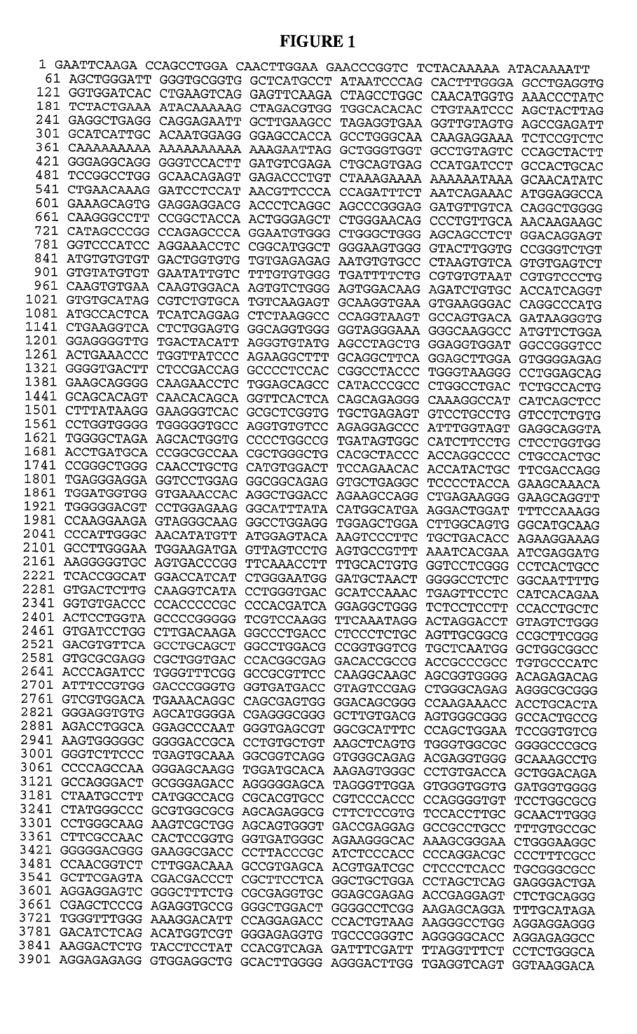

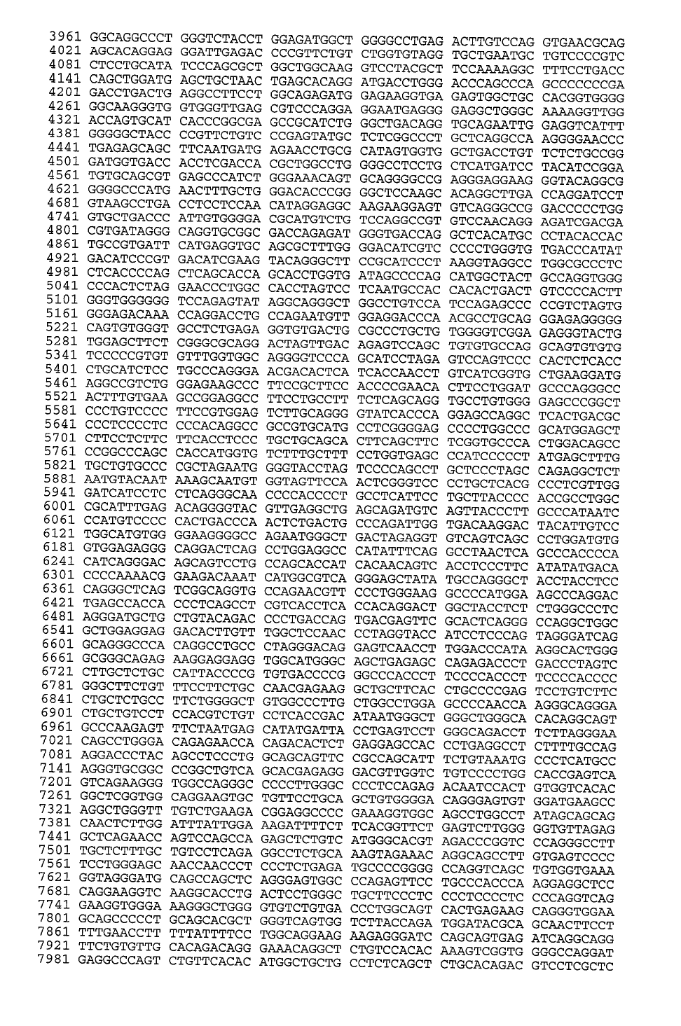

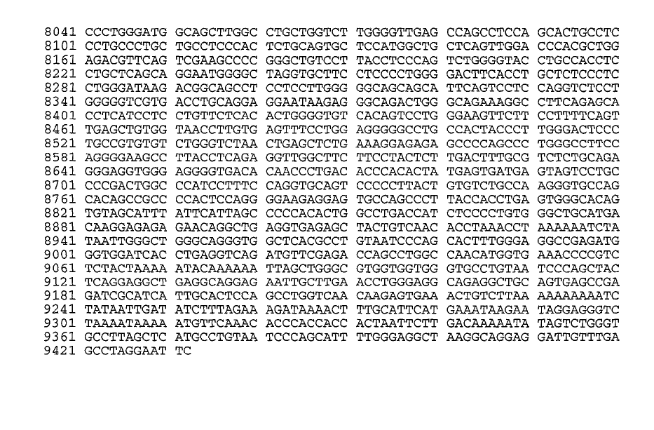

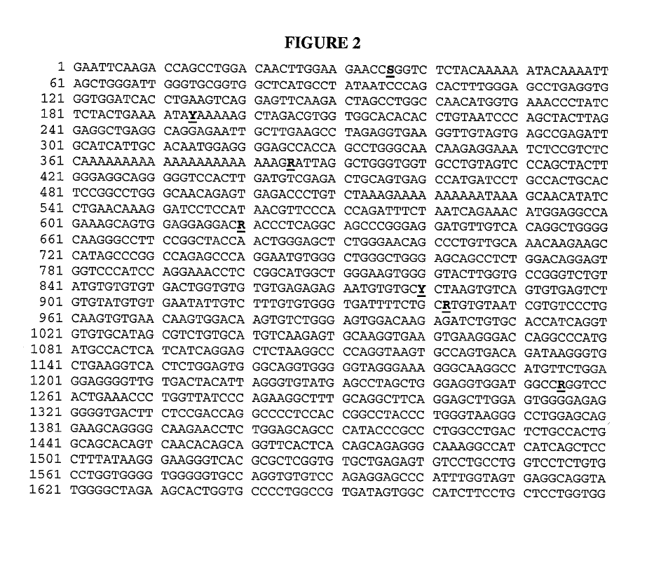

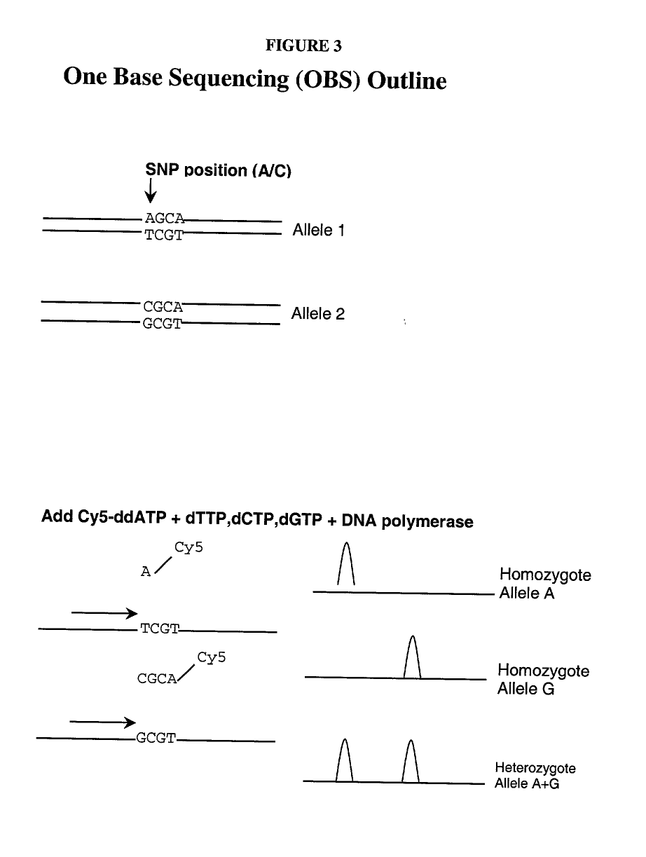

### DETAILED DESCRIPTION OF THE INVENTION

The U.S. patents and publications referenced herein are hereby incorporated by reference.

For the purposes of the invention, certain terms are defined as follows.

“Gene” is defined as the genomic sequence of the CYP2D6 gene.

“Oligonucleotide” means a nucleic acid molecule preferably comprising from about 8 to about 50 covalently linked nucleotides. More preferably, an oligonucleotide of the invention comprises from about 8 to about 35 nucleotides. Most preferably, an oligonucleotide of the invention comprises from about 10 to about 25 nucleotides. In accordance with the invention, the nucleotides within an oligonucleotide may be analogs or derivatives of naturally occurring nucleotides, so long as oligonucleotides containing such analogs or derivatives retain the ability to hybridize specifically within the polymorphic region containing the targeted polymorphism. Analogs and derivatives of naturally occurring oligonucleotides within the scope of the present invention are exemplified in U.S. Pat. Nos. 4,469,863; 5,536,821; 5,541,306; 5,637,683; 5,637,684; 5,700,922; 5,717,083; 5,719,262; 5,739,308; 5,773,601; 5,886,165; 5,929,226; 5,977,296; 6,140,482; WO 00/56746; WO 01/14398, and the like. Methods for synthesizing oligonucleotides comprising such analogs or derivatives are disclosed, for example, in the patent publications cited above and in U.S. Pat. Nos. 5,614,622; 5,739,314; 5,955,599; 5,962,674; 6,117,992; in WO 00/75372, and the like. The term “oligonucleotides” as defined herein includes compounds which comprise the specific oligonucleotides disclosed herein, covalently linked to a second moiety. The second moiety may be an additional nucleotide sequence, for example, a tail sequence such as a polyadenosine tail or an adaptor sequence, for example, the phage M13 universal tail sequence, and the like. Alternatively, the second moiety may be a non-nucleotidic moiety, for example, a moiety which facilitates linkage to a solid support or a label to facilitate detection of the oligonucleotide. Such labels include, without limitation, a radioactive label, a fluorescent label, a chemiluminescent label, a paramagnetic label, and the like. The second moiety may be attached to any position of the specific oligonucleotide, so long as the oligonucleotide retains its ability to hybridize to the polymorphic regions described herein.

A polymorphic region as defined herein is a portion of a genetic locus that is characterized by at least one polymorphic site. A genetic locus is a location on a chromosome which is associated with a gene, a physical feature, or a phenotypic trait. A polymorphic site is a position within a genetic locus at which at least two alternative sequences have been observed in a population. A polymorphic region as defined herein is said to “correspond to” a polymorphic site, that is, the region may be adjacent to the polymorphic site on the 5′ side of the site or on the 3′ side of the site, or alternatively may contain the polymorphic site. A polymorphic region includes both the sense and antisense strands of the nucleic acid comprising the polymorphic site, and may have a length of from about 100 to about 5000 base pairs. For example, a polymorphic region may be all or a portion of a regulatory region such as a promoter, 5′ UTR, 3′ UTR, an intron, an exon, or the like. A polymorphic or allelic variant is a genomic DNA, cDNA, mRNA or polypeptide having a nucleotide or amino acid sequence that comprises a polymorphism. A polymorphism is a sequence variation observed at a polymorphic site, including nucleotide substitutions (single nucleotide polymorphisms or SNPs), insertions, deletions, and microsatellites. Polymorphisms may or may not result in detectable differences in gene expression, protein structure, or protein function. Preferably, a polymorphic region of the present invention has a length of about 1000 base pairs. More preferably, a polymorphic region of the invention has a length of about 500 base pairs. Most preferably, a polymorphic region of the invention has a length of about 200 base pairs.

A haplotype as defined herein is a representation of the combination of polymorphic variants in a defined region within a genetic locus on one of the chromosomes in a chromosome pair. A genotype as used herein is a representation of the polymorphic variants present at a polymorphic site.

Methods of predicting an individual human's capacity to metabolize drugs which are substrates for the CYP2D6 enzyme are encompassed by the present invention. In the methods of the invention, the presence or absence of at least three polymorphic variants of the nucleic acid of SEQ ID NO:2 are detected to determine the individual's haplotype for those variants. Specifically, in a first step, a nucleic acid is isolated from biological sample obtained from the human. Any nucleic-acid containing biological sample from the human is an appropriate source of nucleic acid for use in the methods of the invention. For example, nucleic acid can be isolated from blood, saliva, sputum, urine, cell scrapings, biopsy tissue, and the like. In a second step, the nucleic acid is assayed for the presence or absence of at least three allelic variants of the polymorphic regions of the nucleic acid of SEQ ID NO:2 described above. Specifically, a haplotype is constructed for at least three polymorphic sites in the 5′ regulatory region of the CYP2D6 gene in the method of the invention. The polymorphic sites may be selected from the group consisting of positions 36, 194, and 942 of SEQ ID NO:2; positions 36, 620, and 942 of SEQ ID NO:2; positions 36, 194, and 880 of SEQ ID NO:2; positions 36, 620, and 880 of SEQ ID NO:2; positions 36,194,620, and 942 of SEQ ID NO:2; positions 36,620, 880, and 942 of SEQ ID NO:2, or positions 36, 194, 620, 880, and 942 of SEQ ID NO:2. Preferably, at least three polymorphic sites on each chromosome in the chromosome pair of the human are assayed in the method of the invention, so that the zygosity of the individual for the particular polymorphic variant may be determined.

Any method may be used to assay the nucleic acid, that is, to determine the sequence of the polymorphic region, in this step of the invention. For example, any of the primer extension-based methods, ligase-based sequence determination methods, mismatch-based sequence determination methods, sequencing methods, or microarray-based sequence determination methods described above may be used, in accordance with the present invention. Alternatively, such methods as restriction fragment length polymorphism (RFLP) detection, single strand conformation polymorphism detection (SSCP), PCR-based assays such as the Taqman® PCR System (Applied Biosystems) may be used.

The oligonucleotides of the invention may be used to determine the sequence of the polymorphic regions of SEQ ID NO:2. In particular, oligonucleotides within the scope of the present invention may comprise any of the sequences as set forth in SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO: 14, SEQ ID NO:15, SEQ ID NO:16, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76 and SEQ ID NO:77.

Those of ordinary skill will recognize that oligonucleotides complementary to the polymorphic regions described herein must be capable of hybridizing to the polymorphic regions under conditions of stringency such as those employed in primer extension-based sequence determination methods, restriction site analysis, nucleic acid amplification methods, ligase-based sequencing methods, methods based on enzymatic detection of mismatches, microarray-based sequence determination methods, and the like. The oligonucleotides of the invention may be synthesized using known methods and machines, such as the ABI™3900 High Throughput DNA Synthesizer and the Expedite™8909 Nucleic Acid Synthesizer, both of which are available from Applied Biosystems (Foster City, Calif.).

The oligonucleotides of the invention may be used, without limitation, as in situ hybridization probes or as components of diagnostic assays. Numerous oligonucleotide-based diagnostic assays are known. For example, primer extension-based nucleic acid sequence detection methods are disclosed in U.S. Pat. Nos. 4,656,127; 4,851,331; 5,679,524; 5,834,189; 5,876,934; 5,908,755; 5,912,118; 5,976,802; 5,981,186; 6,004,744; 6,013,431; 6,017,702; 6,046,005; 6,087,095; 6,210,891; WO 01/20039; and the like. Primer extension-based nucleic acid sequence detection methods using mass spectrometry are described in U.S. Pat. Nos. 5,547,835; 5,605,798; 5,691,141; 5,849,542; 5,869,242; 5,928,906; 6,043,031; 6,194,144, and the like. The oligonucleotides of the invention are also suitable for use in ligase-based sequence determination methods such as those disclosed in U.S. Pat. Nos. 5,679,524 and 5,952,174, WO 01/27326, and the like. The oligonucleotides of the invention may be used as probes in sequence determination methods based on mismatches, such as the methods described in U.S. Pat. Nos. 5,851,770; 5,958,692; 6,110,684; 6,183,958; and the like. In addition, the oligonucleotides of the invention may be used in hybridization-based diagnostic assays such as those described in U.S. Pat. Nos. 5,891,625; 6,013,499; and the like.

The oligonucleotides of the invention may also be used as components of a diagnostic microarray. Methods of making and using oligonucleotide microarrays suitable for diagnostic use are disclosed in U.S. Pat. Nos. 5,492,806; 5,525,464; 5,589,330; 5,695,940; 5,849,483; 6,018,041; 6,045,996; 6,136,541; 6,142,681; 6,156,501; 6,197,506; 6,223,127; 6,225,625; 6,229,911; 6,239,273; WO 00/52625; WO 01/25485; WO 01/29259; and the like.

Each of the PCR primer pairs of the invention may be used in any PCR method. For example, a PCR primer pair of the invention may be used in the methods disclosed in U.S. Pat. Nos. 4,683,195; 4,683,202, 4,965,188; 5,656,493; 5,998,143; 6,140,054; WO 01/27327; WO 01/27329; and the like. The PCR pairs of the invention may also be used in any of the commercially available machines that perform PCR, such as any of the GeneAmp® Systems available from Applied Biosystems.

The invention is also embodied in a kit comprising at least three oligonucleotide primer pairs of the invention. Preferably, the kit of the invention comprises at least five oligonucleotide primer pairs, wherein each primer pair is capable of amplifying a different polymorphic region of the nucleic acid of SEQ ID NO:2, said polymorphic regions corresponding to positions 36, 194, 620, 880, and 942 of SEQ ID NO:2. More preferably, the kit of the invention comprises at least four oligonucleotide primer pairs suitable for amplification of polymorphic regions corresponding to positions 36, 194, 620, and 880 of SEQ ID NO:2; or at least four oligonucleotide primer pairs suitable for amplification of polymorphic regions corresponding to positions 36, 194, 620, and 942 of SEQ ID NO:2; or at least four oligonucleotide primer pairs suitable for amplification of polymorphic regions corresponding to positions 36, 620, 880, and 942 of SEQ ID NO:2. Most preferably, the kit of the invention comprises at least three oligonucleotide primer pairs suitable for amplification of polymorphic regions corresponding to positions 36, 194, and 942 of SEQ ID NO:2; or at least three oligonucleotide primer pairs suitable for amplification of polymorphic regions corresponding to positions 36, 194, and 880 of SEQ ID NO:2; or at least three oligonucleotide primer pairs suitable for amplification of polymorphic regions corresponding to positions 36, 620, and 942 of SEQ ID NO:2; or at least three oligonucleotide primer pairs suitable for amplification of polymorphic regions corresponding to positions 36, 620, and 880 of SEQ ID NO:2. This embodiment may optionally further comprise a sequence determination oligonucleotide for detecting a polymorphic variant at any or all of the polymorphic sites corresponding to positions 36, 194, 620, 880, and 942 of SEQ ID NO:2. The kit of the invention may also comprise a polymerizing agent, for example, a thermostable nucleic acid polymerase such as those disclosed in U.S. Pat. Nos. 4,889,818; 6,077,664, and the like. The kit of the invention may also comprise chain elongating nucleotides, such as dATP, dTTP, dGTP, dCTP, and dITP, including analogs of dATP, dTTP, dGTP, dCTP and dITP, so long as such analogs are substrates for a thermostable nucleic acid polymerase and can be incorporated into a growing nucleic acid chain. The kit of the invention may also include chain terminating nucleotides such as ddATP, ddTTP, ddGTP, ddCTP, and the like. In a preferred embodiment, the kit of the invention comprises at least three oligonucleotide primer pairs, a polymerizing agent, chain elongating nucleotides, at least three sequence determination oligonucleotides and at least one chain terminating nucleotide. The kit of the invention may optionally include buffers, vials, microtiter plates, and instructions for use.

In one specific embodiment, the invention provides a kit comprising a pair of oligonucleotide primers suitable for amplifying the polymorphic region corresponding to position 36 of the CYP2D6 gene 5′ flanking region as set forth in SEQ ID NO:2, a primer pair suitable for amplifying the polymorphic region corresponding to position 194 of the CYP2D6 gene 5′ flanking region as set forth in SEQ ID NO:2; a primer pair suitable for amplifying the polymorphic region corresponding to position 942 of the CYP2D6 gene 5′ flanking region as set forth in SEQ ID NO:2; a sequence determination oligonucleotide comprising a sequence selected from the group consisting of SEQ ID NO:3; SEQ ID NO:10; SEQ ID NO:36; SEQ ID NO:37; SEQ ID NO:50; SEQ ID NO:57; SEQ ID NO:64; and SEQ ID NO:71; a sequence determination oligonucleotide comprising a sequence selected from the group consisting of SEQ ID NO:4; SEQ ID NO: 11; SEQ ID NO:38; SEQ ID NO:39; SEQ ID NO:51; SEQ ID NO:58; SEQ ID NO:65; and SEQ ID NO:72; and a sequence determination oligonucleotide comprising a sequence selected from the group consisting of SEQ ID NO:8; SEQ ID NO:15; SEQ ID NO:46; SEQ ID NO:47; SEQ ID NO:55; SEQ ID NO:62; SEQ ID NO:69; and SEQ ID NO:76. The primer pairs of this embodiment are preferably selected from the group consisting of SEQ ID NO:35 and SEQ ID NO: 18 (for amplification of the polymorphic region corresponding to position 36 of SEQ ID NO:2); SEQ ID NO:17 and SEQ ID NO:18; SEQ ID NO:31 and SEQ ID NO:32 (for amplification of the polymorphic region corresponding to position 194 of SEQ ID NO:2); SEQ ID NO:19 and SEQ ID NO:20; and SEQ ID NO:25 and SEQ ID NO:26 (for amplification of the polymorphic region corresponding to position 942 of SEQ ID NO:2.) Alternatively, the invention may be specifically embodied in a kit comprising a pair of oligonucleotide primers suitable for amplifying the polymorphic region corresponding to position 36 of the CYP2D6 gene 5′ flanking region as set forth in SEQ ID NO:2, a primer pair suitable for amplifying the polymorphic region corresponding to position 194 of the CYP2D6 gene 5′ flanking region as set forth in SEQ ID NO:2; a primer pair suitable for amplifying the polymorphic region corresponding to position 880 of the CYP2D6 gene 5′ flanking region as set forth in SEQ ID NO:2; a sequence determination oligonucleotide comprising a sequence selected from the group consisting of SEQ ID NO:3; SEQ ID NO: 10; SEQ ID NO:36; SEQ ID NO:37; SEQ ID NO:50; SEQ ID NO:57; SEQ ID NO:64; and SEQ ID NO:71; a sequence determination oligonucleotide comprising a sequence selected from the group consisting of SEQ ID NO:4; SEQ ID NO:l1; SEQ ID NO:38; SEQ ID NO:39; SEQ ID NO:51; SEQ ID NO:58; SEQ ID NO:65; and SEQ ID NO:72; and a sequence determination oligonucleotide comprising a sequence selected from the group consisting of SEQ ID NO:7; SEQ ID NO:14; SEQ ID NO:44; SEQ ID NO:45; SEQ ID NO:54; SEQ ID NO:61; SEQ ID NO:68; SEQ ID NO:75. The primer pairs of this embodiment are preferably selected from the group consisting of SEQ ID NO:35 and SEQ ID NO:18 (for amplification of the polymorphic region corresponding to position 36 of SEQ ID NO:2); SEQ ID NO: 17 and SEQ ID NO: 18; SEQ ID NO:31 and SEQ ID NO:32 (for amplification of the polymorphic region corresponding to position 194 of SEQ ID NO:2); SEQ ID NO:19 and SEQ ID NO:20; and SEQ ID NO:25 and SEQ ID NO:26 (for amplification of the polymorphic region corresponding to position 880 of SEQ ID NO:2.) In another specific embodiment, the kit of the invention comprises a pair of oligonucleotide primers suitable for amplifying the polymorphic region corresponding to position 36 of the CYP2D6 gene 5′ flanking region as set forth in SEQ ID NO:2, a primer pair suitable for amplifying the polymorphic region corresponding to position 620 of the CYP2D6 gene 5′ flanking region as set forth in SEQ ID NO:2; a primer pair suitable for amplifying the polymorphic region corresponding to position 942 of the CYP2D6 gene 5′ flanking region as set forth in SEQ ID NO:2; a sequence determination oligonucleotide comprising a sequence selected from the group consisting of SEQ ID NO:3; SEQ ID NO: 10; SEQ ID NO:36; SEQ ID NO:37; SEQ ID NO:50; SEQ ID NO:57; SEQ ID NO:64; and SEQ ID NO:71; a sequence determination oligonucleotide comprising a sequence selected from the group consisting of SEQ ID NO:6; SEQ ID NO: 13; SEQ ID NO:42; SEQ ID NO:43; SEQ ID NO:53; SEQ ID NO:60; SEQ ID NO:67; SEQ ID NO:74; and a sequence determination oligonucleotide comprising a sequence selected from the group consisting of SEQ ID NO:8; SEQ ID NO:15; SEQ ID NO:46; SEQ ID NO:47; SEQ ID NO:55; SEQ ID NO:62; SEQ ID NO:69; and SEQ ID NO:76. The primer pairs of this embodiment are preferably selected from the group consisting of SEQ ID NO:35 and SEQ ID NO:18 (for amplification of the polymorphic region corresponding to position 36 of SEQ ID NO:2); SEQ ID NO:27 and SEQ ID NO:28; SEQ ID NO:29 and SEQ ID NO:30 (for amplification of the polymorphic region corresponding to position 620 of SEQ ID NO:2); SEQ ID NO:19 and SEQ ID NO:20; and SEQ ID NO:25 and SEQ ID NO:26 (for amplification of the polymorphic region corresponding to position 942 of SEQ ID NO:2.) In another specific embodiment, the kit of the invention comprises a pair of oligonucleotide primers suitable for amplifying the polymorphic region corresponding to position 36 of the CYP2D6 gene 5′ flanking region as set forth in SEQ ID NO:2, a primer pair suitable for amplifying the polymorphic region corresponding to position 620 of the CYP2D6 gene 5′ flanking region as set forth in SEQ ID NO:2; a primer pair suitable for amplifying the polymorphic region corresponding to position 880 of the CYP2D6 gene 5′ flanking region as set forth in SEQ ID NO:2; a sequence determination oligonucleotide comprising a sequence selected from the group consisting of SEQ ID NO:3; SEQ ID NO: 10; SEQ ID NO:36; SEQ ID NO:37; SEQ ID NO:50; SEQ ID NO:57; SEQ ID NO:64; and SEQ ID NO:71; a sequence determination oligonucleotide comprising a sequence selected from the group consisting of SEQ ID NO:6; SEQ ID NO:13; SEQ ID NO:42; SEQ ID NO:43; SEQ ID NO:53; SEQ ID NO:60; SEQ ID NO:67; SEQ ID NO:74; and a sequence determination oligonucleotide comprising a sequence selected from the group consisting of SEQ ID NO:7; SEQ ID NO:14; SEQ ID NO:44; SEQ ID NO:45; SEQ ID NO:54; SEQ ID NO:61; SEQ ID NO:68; SEQ ID NO:75. The primer pairs of this embodiment are preferably selected from the group consisting of SEQ ID NO:35 and SEQ ID NO:18 (for amplification of the polymorphic region corresponding to position 36 of SEQ ID NO:2); SEQ ID NO:27 and SEQ ID NO:28; SEQ ID NO:29 and SEQ ID NO:30 (for amplification of the polymorphic region corresponding to position 620 of SEQ ID NO:2); SEQ ID NO:19 and SEQ ID NO:20; and SEQ ID NO:25 and SEQ ID NO:26 (for amplification of the polymorphic region corresponding to position 880 of SEQ ID NO:2.) The kit of the invention may optionally include primer pairs for amplification of the polymorphic region corresponding to position 385 of SEQ ID NO:2, such primer pairs being selected from the group consisting of SEQ ID NO:29 and SEQ ID NO:30, and SEQ ID NO:33 and SEQ ID NO:34. The kit of this embodiment also comprises a sequence determination oligonucleotide selected from the group consisting of SEQ ID NO:5; SEQ ID NO:12; SEQ ID NO:40; SEQ ID NO:41; SEQ ID NO:52; SEQ ID NO:59; SEQ ID NO:66; and SEQ ID NO:73.

The kit of the invention may further optionally include primer pairs for amplification of the polymorphic region corresponding to position 1255 of SEQ ID NO:2, such primer pairs being selected from the group consisting of SEQ ID NO:21 and SEQ ID NO:22, and SEQ ID NO:23 and SEQ ID NO:24. The kit of this embodiment also comprises a sequence determination oligonucleotide selected from the group consisting of SEQ ID NO:9; SEQ ID NO:16; SEQ ID NO:48; SEQ ID NO:49; SEQ ID NO:56; SEQ ID NO:63; SEQ ID NO:70; and SEQ ID NO:77.

The examples set forth below are provided as illustration and are not intended to limit the scope and spirit of the invention as specifically embodied therein.

### EXAMPLE 1

### Phenotypes of Study Participants

The study was performed in accordance with the principles stated in the Declaration of Helsinki as reviewed in Tokyo 1975 and Venice 1983, Hong Kong 1989 and Somerset West 1996. Subjects were preferably not related to each other. Based on questioning, individuals having one of the following were excluded: a medical condition judged to influence liver function or requiring pharmacological treatment; any on-going disease; intake of any drug, except oral contraceptives, during one week prior to the study; breast-feeding or pregnancy. No physical examination was performed. For these experiments, a single oral dose of 10 mg debrisoquine (Declinax, Hoffman-LaRoche) was taken in the evening before bed-time. The bladder was emptied before drug intake. All urine was then collected overnight (about 8 hours) A single blood sample was collected 3 hours after drug intake.

In the first part of the study, 88 samples (Swedish Caucasians) were selected as set forth in Table 1, on the basis of the following assumptions: if the distribution of an unknown polymorphism will be 25% for a homozygote, a sample size of approximately 40 “UEM” will be able to detect an increase in this specific genotype (homozygote) by 28% (α=5% (two-tailed), power=80%). If it is assumed that the distribution of an unknown polymorphism will be 10% for a homozygote, a sample size of approximately 40 “UEM” will be able to detect an increase in this specific genotype (homozygote) by 21% (α=5% (two-tailed), power=80%).

Individuals with UEM phenotype caused by CYP2D6-gene duplication were excluded. Individuals with known defective alleles, i.e. CYP2D6*3, CYP2D6*4 and CYP2D6*5 were excluded. CYP2D6*6 was also excluded where data was available (and due to its low allele frequency among Caucasians (1.8%) additional *6 genotyping was not applied as a standard procedure). However, a few extra samples genotyped for any of the alleles mentioned above were included as outlier controls.

TABLE 1

| # of samples | MR | Phenotype |
|---|---|---|
| 47 | <0.2 | “UEM” |
| 26 | 0.2-0.8 | “fast EM” |
| 11 | 0.8-12.6 | “slow EM” |
| 4 | >12.6 | “PM” |

The first part of the study resulted in identification of seven SNPs in the 5′ flanking region of the CYP2D6 gene. Oligonucleotides containing these SNPs are shown in Table 2.

TABLE 2

| Poly- | | | |
|---|---|---|---|
| morphic | | | Nucleotide |
| Site | Sequence | | change |
| 36 | SEQ ID NO:3: | GAACCCGGTCT | C variant |
| (−1496) | SEQ ID NO:10: | GAACCGGGTCT | G variant |
| 194 | SEQ ID NO:4: | AAAATACAAAAAG | C variant |
| (−1338) | SEQ ID NO:11: | AAAATATAAAAAG | T variant |
| 385 | SEQ ID NO:5: | AAAAAGAATTAGG | A variant |
| (−1147) | SEQ ID NO:12: | AAAAAGGATTAGG | G variant |
| 620 | SEQ ID NO:6: | AGGACGACCCT | G variant |
| (−912) | SEQ ID NO:13: | AGGACAACCCT | A variant |
| 880 | SEQ ID NO:7: | TGTGCCCTAAG | C variant |
| (−652) | SEQ ID NO:14: | TGTGCTCTAAG | T variant |
| 942 | SEQ ID NO:8: | TCTGCGTGTGT | G variant |
| (−590) | SEQ ID NO:15: | TCTGCATGTGT | A variant |
| 1255 | SEQ ID NO:9: | TGGCCGGGTCC | G variant |
| (−277) | SEQ ID NO:16: | TGGCCAGGTCC | A variant |

In the second part of the study, samples with a more normative phenotypic distribution were used. Also, no exclusion of individuals with known defective alleles or duplications was done. Table 3 sets forth the phenotypic distribution of 144 samples used in the second part of this study.

TABLE 3

| # of samples | MR | Phenotype |
|---|---|---|
| 15 | <0.2 | “UEM” |
| 78 | 0.2-0.8 | “fast EM” |
| 33 | 0.81-12.6 | “slow EM” |
| 18 | >12.6 | “PM” |

### EXAMPLE 2

### CYP2D6 Genetic Analysis

White blood cells isolated from a blood sample drawn from the brachial vein serve as the source of the genomic DNA for the analyses. The DNA was extracted by guanidine thiocyanate method or QlAamp Blood Kit (QIAGEN, Venlo, The Netherlands). The genes included in the study were amplified by PCR and the DNA sequences were determined by the technology most suitable for the specific fragment. All genetic analyses were performed according to Good Laboratory Practice and Standard Operating Procedures. Case Report Forms were designed and used for clinical and genetic data collection. Data was entered and stored in a relational database at Gemini Genomics AB, Uppsala. To secure consistency between the Case Report Forms and the database, data was checked either by double data entry or proofreading. After a Clean File was declared the database was protected against changes. By using the program Stat/Transfer™ the database was transferred to SAS data sets. The SAS™ system was used for tabulations and statistical evaluations. Genotypes and haplotypes were correlated against the metabolic ratio.

PCR-fragments were amplified with TaqGOLD polymerase (Applied Biosystems) using Robocycler (Stratagene) or GeneAmp PCR system 9700 (Applied Biosystems). Preferentially, the amplified fragments were 300-400 bp, and the region to be read did not exceed 300 bp for full sequencing and did not exceed 60 bp for One Base Sequencing (OBS). PCR reactions were carried out according to the basic protocol set forth in Table 4, with modifications as indicated in Table 5 for specific primer pairs, which are shown in Table 6. For the GeneAmp PCR 9700 machine the profile used was 10 minutes at 95°, 40×(45 seconds at 90°, 45 seconds at 60°, 45 seconds at 72°), 5 minutes at 72° and 22° until removed.

TABLE 4

| Solution | Stock Concentration | | PCR (μl) |
|---|---|---|---|
| H₂O | | | 33.2 |
| PCR buffer | 10x | | 5.0 |
| MgCl₂ | 25 | mM | 2.0 |
| dNTP | 2.5 | mM | 2.5 |
| primer 1 | 10 | μM | 1.0 |
| primer 2 | 10 | μM | 1.0 |
| Taq-gold | 5 | μ/μl | 0.3 |
| polymerase | | | |
| DNA sample | 2 | ng/μl | 5.0 |
| TOTAL | | | 50.0 |

TABLE 5

| SEQ ID | Polymorphic | | |
|---|---|---|---|
| NO:s | Site | Modification from basic protocol (Table 3) | SNP Detection method |
| 35, 18 | 36 | 640 annealing temperature | Full sequencing & OBS |
| 35, 18 | 36 | 620 annealing temperature | OBS |
| 17, 18 | 194 | 620 annealing temperature | Full sequencing & OBS |
| 31, 32 | 194 | 580 annealing temperature, 50 cycles | Full sequencing |
| 33, 34 | 385 | 3 μl MgCl₂, 580 annealing temperature | Full sequencing & OBS |
| 29, 30 | 385 & 620 | None | Full sequencing |
| 27, 28 | 620 | 620 annealing temperature, 50 cycles | Full sequencing & OBS |
| 19, 20 | 880 & 942 | 620 annealing temperature | Full sequencing & OBS |
| 25, 26 | 880 & 942 | None | Full sequencing & OBS |
| 21, 22 | 1255 | 3 μl MgCl₂ | Full sequencing |
| 23, 24 | 1255 | 620 annealing temperature | Full sequencing & OBS |

TABLE 6

| | |
|---|---|
| Polymorphic | |
| Site | Primer Pair |

| | | |
|---|---|---|
| 194 | SEQ ID NO:17; | AAATACAAAATTAGCTGGGATTG |
| | SEQ ID NO:18: | GAGACGGAGATTTCCTCTTGT |
| 880 & 942 | SEQ ID NO:19: | CCTTCCGGCTACCAACTG |
| | SEQ ID NO:20: | TTGCAGGGACACGATTACAC |
| 1255 | SEQ ID NO:21: | TAAGGGTGCTGAAGGTCACTC |
| | SEQ ID NO:22: | GGGCTGCTCCAGAGGTTC |
| 1255 | SEQ ID NO:23: | CCAGGTAAGTGCCAGTGACA |
| | SEQ ID NO:24: | AGCTCCTGAAGCCTGCAAAG |
| 880 & 942 | SEQ ID NO:25: | GCCAGAGCCCAGGAATGT |
| | SEQ ID NO:26: | GCCTTGCCCTTTCCCTAC |
| 620 | SEQ ID NO:27: | AGAAACATGGAGGCCAGAA |
| | SEQ ID NO:28: | GTTTCCTGGATGGGACCAC |
| 385 & 620 | SEQ ID NO:29: | AGCCTAGAGGTGAAGGTTGTAG |
| | SEQ ID NO:30 | CTTGCCCCAGCCTGTGA |
| 194 | SEQ ID NO:31: | AAAAAATACAAAATTAGCTGGGATT |
| | SEQ ID NO:32: | TTTTTTTTTGGAGACGGAGAT |
| 385 | SEQ ID NO:33: | TTCTTTAGACAGGGTCTCACTCT |
| | SEQ ID NO.34: | GGGCAACAAGAGGAAATCT |
| 36 | SEQ ID NO:35: | GCCTGGACAACTTGGAAGA |
| | SEQ ID NO:18: | GAGACGGAGATTTCCTCTTGT |

The sequences of Table 8 represent the 5′-sequence to the polymorphic sites on the coding (sense) strand (SEQ ID NO:s 50-56) and non-coding (anti-sense) strand (SEQ ID NO:s 57-63). The underlined letter indicates polymorphic position in the sequence context. Numbers inside brackets are calculated from the transcriptional start. All sequences are shown in 5′ to 3′ direction.

TABLE 8

| | | |
|---|---|---|
| Polymorphic | | |
| Site | Sequence | Note |

| | | | |
|---|---|---|---|
| 36 | SEQ ID NO:50: | ACTTGGAAGAA | Sense 5′ |
| (−1496) | SEQ ID NO:57: | TTCTTCCAAGT | Antisense 5′ |
| 194 | SEQ ID NO:51: | TCTACTGAAAA | Sense 5′ |
| (−1338) | SEQ ID NO:58: | TTTTCAGTAGA | Antisense 5′ |
| 385 | SEQ ID NO:52: | CCAAAAAAAAAAAAAAAAAAAAAAG | Sense 5′ |
| (−1147) | SEQ ID NO:59: | CTTTTTTTTTTTTTTTTTTTTTTGG | Antisense 5′ |
| 620 | SEQ ID NO:53: | AGTGGAGGAGG | Sense 5′ |
| (−912) | SEQ ID NO:60: | CCTCCTCCACT | Antisense 5′ |
| 880 | SEQ ID NO:54: | AGAGAATGTGT | Sense 5′ |
| (−652) | SEQ ID NO:61: | ACACATTCTCT | Antisense 5′ |
| 942 | SEQ ID NO:55: | GGTGATTTTCT | Sense 5′ |
| (−590) | SEQ ID NO:62: | AGAAAATCACC | Antisense 5′ |
| 1255 | SEQ ID NO:56: | GAGGTGGATGG | Sense 5′ |
| (−277) | SEQ ID NO:63: | CCATCGACCTC | Antisense 5′ |

The sequences of Table 9 represent the 3′-sequence to the polymorphic sites on the non-coding (anti-sense) strand (SEQ ID NO:s 64-70) and the coding (sense) strand (SEQ ID NO:s 71-77). Underlined letter indicates polymorphic position in the sequence context. Numbers inside brackets are calculated from the transcriptional start. All sequences are shown in 5′ to 3′ direction.

TABLE 9

| Polymorphic | | | |
|---|---|---|---|
| Site | Sequence | | Note |
| 36 | SEQ ID NO:64: | TTTTGTAGAGA | Antisense 3′ |
| (−1496) | SEQ ID NO:71: | TCTCTACAAAA | Sense 3′ |
| 194 | SEQ ID NO:65: | CGTCTAGCTTT | Antisense 3′ |
| (−1338) | SEQ ID NO:72: | AAAGCTAGACG | Sense 3′ |
| 385 | SEQ ID NO:66: | CACCCAGCCTA | Antisense 3′ |
| (−1147) | SEQ ID NO:73: | TAGGCTGGGTG | Sense 3′ |
| 620 | SEQ ID NO:67: | GCTGCCTGAGG | Antisense 3′ |
| (−912) | SEQ ID NO:74: | CCTCAGGCAGC | Sense 3′ |
| 880 | SEQ ID NO:68: | CACTGACACTT | Antisense 3′ |
| (−652) | SEQ ID NO:75: | AAGTGTCAGTG | Sense 3′ |
| 942 | SEQ ID NO:69: | ACACGATTACA | Antisense 3′ |
| (−590) | SEQ ID NO:76: | TGTAATCGTGT | Sense 3′ |
| 1255 | SEQ ID NO:70: | GTTTCAGTGGA | Antisense 3′ |
| (−277) | SEQ ID NO:77: | TCCACTGAAAC | Sense 3′ |

### EXAMPLE 3

### Haplotype and Genotype Analyses

Haplotype analysis could be performed on a total of 232 individuals. This analysis was performed using software based on maximum likelihood methodology and using the EM algorithm of Excoffier et al. (1995), Mol Biol Evol. 12:921-927. In total 5 likely haplotypes were identified by the program. One of these occurred only six times in the study population and has been excluded from the study due to its low frequency. The characterization of each haplotype is presented in Table 10, and the frequency of each haplotype is set forth in Table 11. From the haplotype information two different kinds of variables were created: one variable was formed as a haplotype combination variable (HTYPE). This variable has the value H1/H2 when the subject has haplotypes 1 and 2, etc. Variables H1, H2, H3 and H4 are haplotype annotations that denote the number of copies of that particular haplotype for the subject, e.g., for a subject with haplotype H1/H2 the variables H1, H2, H3 and H4 will be 1, 1, 0 and 0, respectively. Each of these variables can thus take on the values 0, 1 or 2. Only the four most frequent haplotypes were considered when those variables were formed.

TABLE 10

| | Nucleotide at polymorphic position: | | | | | | |
|---|---|---|---|---|---|---|---|
| Haplotype | 36 | 194 | 385 | 620 | 880 | 942 | 1255 |
| M33388 | C | C | A | G | C | G | G |
| (GenBank) | | | | | | | |
| SEQ ID NO:1 | | | | | | | |
| H1 | C | C | A | G | C | G | G |
| (CCAGCGG) | | | | | | | |
| H2 | G | C | G | G | T | A | G |
| (GCGGTAG) | | | | | | | |
| H3* | C | C | G | G | T | A | G |
| (CCGGTAG) | | | | | | | |
| H4* | C | T | G | A | C | G | G |
| (CTGACGG) | | | | | | | |

TABLE 11

| | Haplotype | | | | |
|---|---|---|---|---|---|
| Haplotype | frequency | P-value (Sp) | Note | | |
| H1 | 46% | 0.0001 | H1/H1 | n = 53 | mr50 = 0.22 |
| | | | H1/— | n = 108 | mr50 = 0.375 |
| | | | —/— | n = 71 | mr50 = 0.87 |
| H2 | 27% | 0.0001 | H2/H2 | n = 19 | mr50 = 0.3 |
| | | | H2/— | n = 88 | mr50 = 0.335 |
| | | | —/— | n = 125 | mr50 = 0.56 |
| H3 | 8% | 0.0012 | H3/H3 | n = 3 | mr50 = 2.08 |
| | | | H3/— | n = 31 | mr50 = 0.64 |
| | | | —/— | n = 198 | mr50 = 0.34 |
| H4 | 17% | 0.0001 | H4/H4 | n = 16 | mr50 = 88 |
| | | | H4/— | n = 49 | mr50 = 0.86 |
| | | | —/— | n = 167 | mr50 = 0.27 |

Table 11 also sets forth the statistical p-values (Spearman correlation) between CYP2D6 haplotypes H1-H4 and mr(debrisoquine), where mr50 is an abbreviation for metabolic ratio of the 50^{th} percentile.

Table 12 sets forth a summary of the predictive haplotypes found in the study 15 described in Examples 1 and 2.

TABLE 12

| Haplotype | Metabolic capacity | Note |
|---|---|---|
| H1 | UEM & EM | H1/H2 is faster (UEM/EM) |
| H2 | UEM & EM | H1/H2 is faster (UEM/EM) |
| H3 | IM | |
| H4 | PM | In 99% LD with CYP2D6*4 |
| | | (80 samples/81 samples) |

Table 13 shows CYP2D6 genotype markers for haplotype combinations and their predicted metabolic ratios based on 232 samples. It should be noted that the method of the invention may use detection of only three SNPs in the 5′ flanking region of the CYP2D6 gene, since position 2D6: 194 can be replaced with position 2D6:620, and position 2D6:942 with position 2D6:880 with the same resolution power as shown in Table 13.

TABLE 13

| CYP2D6 genotype | | | | | | % of haplotypes | MR-range |
|---|---|---|---|---|---|---|---|
| 2D6:36 | 2D6:194 | 2D6:942 | HTYPE | Marker for | MR (Debr) | in MR-range | (min-max) |
| C/G | C | A/G | H1/H2 | UEM/EM | <0.4 | 81% (52/64) | 0.06-1.04 |
| C | C | G | H1/H1 | UEM & EM | <0.8 | 89% (49/55) | 0.03-110 |
| G | C | A | H2/H2 | UEM & EM | <0.8 | 89% (17/19) | 0.13-1.44 |
| C/G | C | A | H2/H3 | EM & IM | 0.2-7.0 | 86% (6/7) | 0.13-2.29 |
| C | C | A/G | H1/H3 | EM | 0.4-2.0 | 74% (14/19) | 0.08-2.40 |
| C/G | C/T | A/G | H2/H4 | EM | 0.4-2.0 | 82% (14/17) | 0.33-3.70 |
| C | C/T | G | H1/H4 | EM & IM | 0.4-7.0 | 74% (20/27) | 0.18-143 |
| C | C | A | H3/H3 | IM | 0.8-7.0 | 100% (3/3) | 0.85-5.17 |
| C | C/T | A/G | H3/H4 | IM | 0.8-7.0 | 100% (5/5) | 1.46-6.54 |
| C | T | G | H4/H4 | PM | >12.6 | 100% (16/16) | 26.3-236 |

While the invention has been described in terms of the specific embodiments set forth above, those of skill will recognize that the essential features of the invention may be varied without undue experimentation and that such variations are within the scope of the appended claims.

1. A method for determining a human's capacity to metabolize a substrate of a CYP2D6 enzyme, said method comprising the steps of:
a) isolating single stranded nucleic acids from the human, said nucleic acids encoding 5′ flanking regions of CYP2D6 genes present on each homologous chromosome 22 of the human, wherein said region is represented by a sequence as set forth in SEQ ID NO:2; and
b) detecting at least three polymorphisms within the region, wherein the polymorphisms are selected from the group consisting of nucleotides present at polymorphic sites represented by positions 36,194, and 942of SEQ ID NO:2; nucleotides present at polymorphic sites represented by positions 36, 620, and 942 of SEQ ID NO:2; nucleotides present at polymorphic sites represented by positions 36, 194, and 880 of SEQ ID NO:2; nucleotides at polymorphic sites represented by positions 36, 620, and 880 of SEQ ID NO:2; nucleotides at polymorphic sites represented by positions 36, 194, 620, and 880 of SEQ ID NO:2; nucleotides at polymorphic sites represented by positions 36, 194, 620, and 942 of SEQ ID NO:2; nucleotides at polymorphic sites represented by positions 36, 620, 880, and 942 of SEQ ID NO:2; and nucleotides at polymorphic sites represented by positions 36, 194, 620, 880, and 942 of SEQ ID NO:2.

2. A sequence determination oligonucleotide suitable for detecting polymorphic sites in a 5′ flanking region of a CYP2D6 gene, said oligonucleotide comprising a sequence selected from the group consisting of SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16; SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76 and SEQ ID NO:77.

3. An oligonucleotide primer pair suitable for amplifying a 5′ flanking region of a CYP2D6 gene, said primer pair having sequences selected from the group consisting of: SEQ ID NO:17 and SEQ ID NO:18; SEQ ID NO:19 and SEQ ID NO:20; SEQ ID NO:21 and SEQ ID NO:22; SEQ ID NO:23 and SEQ ID NO:24; SEQ ID NO:25 and SEQ ID NO:26; SEQ ID NO:27 and SEQ ID NO:28; SEQ ID NO:29 and SEQ ID NO:30; SEQ ID NO:31 and SEQ ID NO:32; SEQ ID NO:33 and SEQ ID NO:34; and SEQ ID NO:35 and SEQ ID NO:18.

4. A kit comprising at least three oligonucleotide primer pairs suitable for amplification of polymorphic regions corresponding to positions 36, 194, and 942 of SEQ ID NO:2; or at least three oligonucleotide primer pairs suitable for amplification of polymorphic regions corresponding to positions 36, 194, and 880 of SEQ ID NO:2; or at least three oligonucleotide primer pairs suitable for amplification of polymorphic regions corresponding to positions 36, 620, and 942 of SEQ ID NO:2; or at least three oligonucleotide primer pairs suitable for amplification of polymorphic regions corresponding to positions 36, 620, and 880 of SEQ ID NO:2.

5. The kit of claim 4, wherein the primer pairs are suitable for amplification of polymorphic regions corresponding to positions 36, 194, and 942 of SEQ ID NO:2; further comprising:
i) a sequence determination oligonucleotide comprising a sequence selected from the group consisting of SEQ ID NO:3; SEQ ID NO: 10; SEQ ID NO:36; SEQ ID NO:37; SEQ ID NO:50; SEQ ID NO:57; SEQ ID NO:64; and SEQ ID NO:71;
ii) a sequence determination oligonucleotide comprising a sequence selected from the group consisting of SEQ ID NO:4; SEQ ID NO:11; SEQ ID NO:38; SEQ ID NO:39; SEQ ID NO:51; SEQ ID NO:58; SEQ ID NO:65; and SEQ ID NO:72; and
iii) a sequence determination oligonucleotide comprising a sequence selected from the group consisting of SEQ ID NO:8; SEQ ID NO:15; SEQ ID NO:46; SEQ ID NO:47; SEQ ID NO:55; SEQ ID NO:62; SEQ ID NO:69; and SEQ ID NO:76.

6. The kit of claim 4, wherein the primer pairs are suitable for amplification of polymorphic regions corresponding to positions 36, 194, and 880 of SEQ ID NO:2; further comprising:
i) a sequence determination oligonucleotide comprising a sequence selected from the group consisting of SEQ ID NO:3; SEQ ID NO:10; SEQ ID NO:36; SEQ ID NO:37; SEQ ID NO:50; SEQ ID NO:57; SEQ ID NO:64; and SEQ ID NO:71;
ii) a sequence determination oligonucleotide comprising a sequence selected from the group consisting of SEQ ID NO:4; SEQ ID NO: 1; SEQ ID NO:38; SEQ ID NO:39; SEQ ID NO:51; SEQ ID NO:58; SEQ ID NO:65; and SEQ ID NO:72; and
iii) a sequence determination oligonucleotide comprising a sequence selected from the group consisting of SEQ ID NO:7; SEQ ID NO: 14; SEQ ID NO:44; SEQ ID NO:45; SEQ ID NO:54; SEQ ID NO:61; SEQ ID NO:68; SEQ ID NO:75.

7. The kit of claim 4, wherein the primer pairs are suitable for amplification of polymorphic regions corresponding to positions 36, 620, and 942 of SEQ ID NO:2; further comprising:
i) a sequence determination oligonucleotide comprising a sequence selected from the group consisting of SEQ ID NO:3; SEQ ID NO: 10; SEQ ID NO:36; SEQ ID NO:37; SEQ ID NO:50; SEQ ID NO:57; SEQ ID NO:64; and SEQ ID NO:71;
ii) a sequence determination oligonucleotide comprising a sequence selected from the group consisting of SEQ ID NO:6; SEQ ID NO:13; SEQ ID NO:42; SEQ ID NO:43; SEQ ID NO:53; SEQ ID NO:60; SEQ ID NO:67; SEQ ID NO:74; and
iii) a sequence determination oligonucleotide comprising a sequence selected from the group consisting of SEQ ID NO:8; SEQ ID NO:15; SEQ ID NO:46; SEQ ID NO:47; SEQ ID NO:55; SEQ ID NO:62; SEQ ID NO:69; and SEQ ID NO:76.

8. The kit of claim 4, wherein the primer pairs are suitable for amplification of polymorphic regions corresponding to positions 36, 620, and 880 of SEQ ID NO:2; further comprising:
i) a sequence determination oligonucleotide comprising a sequence selected from the group consisting of SEQ ID NO:3; SEQ ID NO: 10; SEQ ID NO:36; SEQ ID NO:37; SEQ ID NO:50; SEQ ID NO:57; SEQ ID NO:64; and SEQ ID NO:71;
ii) a sequence determination oligonucleotide comprising a sequence selected from the group consisting of SEQ ID NO:6; SEQ ID NO: 13; SEQ ID NO:42; SEQ ID NO:43; SEQ ID NO:53; SEQ ID NO:60; SEQ ID NO:67; SEQ ID NO:74; and
iii) a sequence determination oligonucleotide comprising a sequence selected from the group consisting of SEQ ID NO:7; SEQ ID NO: 14; SEQ ID NO:44; SEQ ID NO:45; SEQ ID NO:54; SEQ ID NO:61; SEQ ID NO:68; SEQ ID NO:75.
